(19) United States
(12) Patent Application Publication
(10) Pub. No.: US 2026/0215691 A1
(43) Pub. Date: Jul. 30, 2026

(54) METHOD AND SYSTEM FOR PATIENT THERMOREGULATION

(71) Applicant: EverSilkSkin LLC, Sacramento, CA (US)

(72) Inventors: Antonio De Jesus Alvarado Garcia, Chula Vista, CA (US); Sarah Saleem, Islamabad (PK)

(73) Assignee: EverSilkSkin LLC, Sacramento, CA (US)

(21) Appl. No.: 19/458,663

(22) Filed: Jan. 23, 2026

Related U.S. Application Data
(60) Provisional application No. 63/750,769, filed on Jan. 28, 2025.

(51) Int. Cl. A61B 5/01; A61B 5/00; A61B 5/024; A61B 5/08; A61F 7/00

(52) U.S. Cl. CPC A61B 5/01; A61B 5/024; A61B 5/0816; A61B 5/7282; A61B 5/746; A61F 7/007; A61B 2560/0242; A61B 2560/0462; A61B 2562/0271; A61F 2007/0075

(57) ABSTRACT

A method and system is disclosed for incorporating a deep learning framework in a HIPPA complaint data workflow to determine a stringent baseline core temperature. The method can infer an onset of hypothermia and hypothermia from this baseline, and provide an option to rapidly assist medical staff in patient thermoregulation using low-voltage sensors applied to the patient. The method incorporates the computational power of neural networks that are capable of computing combinations of a large number of variables to predict future temperature behavior and preemptively treat or prevent temperature related conditions.

### CROSS-REFERENCES TO RELATED APPLICATIONS

This application claims priority from U.S. Provisional Application No. 63/750,769, filed Jan. 28, 2025, the contents of which is fully incorporated by reference in its entirety.

### BACKGROUND

The present invention relates generally to the field of medical services, and more particularly to a method and system for regulating and controlling a patient's core body temperature.

In the healthcare industry the importance of ascertaining and controlling a patient's temperature is paramount. According to the National Library of Medicine, there exists an indicator pattern where the development of sepsis is in tandem with an increase of the core body temperature. This is most notable in a recent study correlating the mutation rate of pathogenic microbes to temperature changes residing in the normal body temperature range. However, the hypothesis for temperature deviation and sepsis has been researched and it was found that a 1° C. positive shift in the base body temperature led patients to sequential organ failures. This has facilitated septicemia to remain a top cause in hospital deaths: amongst 1.7 million adults who develop sepsis, 270,000 will die as a result. Out of these cases, the World Health Organization reported that around 1.5% of hospitalized patients develop the condition due to complications arising from healthcare delivery. In addition, studies on nursing complications suggest that hospitals continue to remain understaffed and patients remain under monitored, while a temperature value standardizing fever continues to be unagreeable. While these statistics are insufficiently salient when juxtaposed to current mortality rate factors, the combination of healthcare delivery, nursing burden, and the rise of antibiotic resistance complications delineates a trend toward involuntary health conditions acquired by temperature dysregulation from a hospital visit.

Temperature remains a critical parameter among the seven vital signs monitored in clinical settings. Traditionally, medical professionals consider five major vital signs, including temperature, to assess patient health. This makes core temperature of the human body a fundamental indicator of wellness in healthcare, where even minor fluctuations are capable of both triggering and signaling various illnesses, establishing it as a key diagnostic metric in perioperative care. In the context of disease control, epidemics, and pandemics, monitoring body temperature is the first line of defense in infection prevention. This was exemplified during the recent SARS19 global pandemic, where temperature screenings played a pivotal role in pandemic preparedness, specifically determining health from compromised individuals solely based on temperature degree differences. However, in an isolated medical or clinical setting, it is crucial for nurses to be able to visualize temperature trends before the symptoms of abnormal core temperature become apparent. When this is combined with a limited amount of heating and cooling resources in healthcare settings, where the temperature can change as an effect or cause of multifaceted processes, the prevention of hypothermia or hyperthermia remains elusive.

In addition, monitoring and controlling core body temperature to prevent hypothermia and hyperthermia presents significant challenges for hospital staff. In their daily routines, nurses often manually record patient temperatures, storing this data in an Electronic Health Record (EHR) system. While useful for historical analysis, this method provides only static temperature snapshots, failing to reveal emerging trends or predict future temperature fluctuations. Primarily driven by the complexity of core body temperature regulation, understanding of core temperature predictors forms part of a multilayered physiological and environmental network, making non-invasive temperature predictions nearly an impossible task. This is further exacerbated by the emergence of the nursing burden that leads to cognitive overload in high-pressure healthcare environments and which can cause staff to lack continuous temperature monitoring for a patient.

Additionally, to prevent the worsening of a patient's condition, it remains imperative to alert a healthcare facility to act preemptively in precedence of the first symptoms of hypothermia and hyperthermia. However, while not all temperature dysregulation is a consequence of fever, all temperature dysregulation from an unspecified fever temperature point can lead to the worsening of temperature-related complications in an upcoming antibiotic resistance age.

Current core body temperature monitoring and controlling systems, while practical, face significant limitations that hinder their effectiveness in critical care settings. One major issue is the inaccuracy of core temperature estimation, as these systems rely solely on classical algorithms using skin temperature data points, which can be easily influenced by eclectic factors such as ambient temperature, body composition, airflow, or clothing in a general wellness institution. This reliance leads to inconsistent readings of temporal deviations that are not accounted for, and lead to a delay to reflect the body's internal core temperature in real time. In situations such as post-surgeries or in intensive care units, even minor inaccuracies in temperature can result in serious complications. Additionally, these systems often fail to provide real-time adaptive regulation, as they only react after a deviation from the preset core temperature occurs. This further delay in corrective action increases the risk of under-or over-correcting temperature fluctuations, causing further instability in the patient's condition. Lastly, the use of general cooling methods, such as blankets, fans and liquid elements hinder the delicate balance between heat gain and heat loss of the skin during core temperature homeostasis, while creating a venue for moisture to assist in the growth of certain microbes, which could prove to be pathogenic. The combination of one or more of these elements create a nursing burden by limiting the physical room and skin space available for a nurse to interact with a patient.

Another critical limitation in current systems is their lack of personalization and reliance on narrow data sets. These systems apply the same temperature control mechanisms across all users, regardless of individual physiological differences such as age, body composition, actual time or underlying health conditions. This one-size-fits-all approach can lead to discomfort or even medical complications for patients with unique thermoregulatory needs in their visit to a general wellness institution. Moreover, without multi-sensor integration, the systems cannot fully comprehend the complexity of the body's thermoregulatory processes, making their estimation of thermoregulation prediction non-existent or reliant on only local blood perfusion under a single blood vessel to understand a correlation between peripheral temperature to core temperature. In addition, bulky devices or outdated technologies may hinder the movement of professionals when faced with a bustling, compact space.

From a control systems perspective, all of these technologies operate in a reactive mode, with corrections initiated only after deviations from the target temperature have been detected. This delay, compounded by the nonlinear dynamics and time-varying properties of human thermoregulation, often leads to suboptimal transient and steady-state responses. Specifically, PID controllers are prone to overshoot and oscillations, particularly when confronted with nonlinearities such as the body's heat transfer coefficients or variable metabolic rates. These PID controllers activate or deactivate available actuators such as thermoelectric coolers (TECs), high-voltage Peltier modules, phase-change fluid systems, resistive heating films, and mechanical fans embedded in a medium as a response to temperature fluctuations.

While these systems effectively maintain basic homeostasis, they lack the sophistication for precise and adaptive thermal management in complex environments due to maximized surface area of cooling or heating exchange. As a result, they tend to respond reactively to temperature deviations using bulky and imprecise mechanisms, without dynamically adapting to real-time physiological or environmental changes.

Finally, current systems suffer from a lack of advanced technologies like machine learning or deep learning frameworks which could revolutionize temperature management by creating a closer estimate and providing the foundation to predict core temperature of the human body. Without predictive capabilities, these systems are purely reactive, adjusting temperature only after it deviates from the set range rather than anticipating changes based on historical data or trends; this absence of prediction hampers the system's ability to maintain consistent core body temperature, especially during quickly fluctuating environmental or physiological conditions. Additionally, limited integration with Electronic Health Record data or other personalized medical data prevents these systems from tailoring interventions based on a patient's unique physiological state. Combined with bulky designs, energy inefficiency, and narrow data sets, our current temperature estimating systems fall short in providing non-invasive, individualized and precise temperature thermoregulation assistance through an understanding of positive and negative heat flux of the integumentary system during core temperature homeostasis.

In summary, current core temperature monitoring and controlling systems remain inadequate as they are unable to estimate individual core temperature using robust neural networks that take in account the height, weight, age, race, sex, the time of day, and the length of stay in their temperature dependent parameters, while understanding comprehensive heat flux trends during thermoregulation. The present invention seeks to improve on the current methods and overcome the drawbacks of traditional temperature monitoring.

### SUMMARY OF THE INVENTION

Amid the aforementioned limitations and findings to patient wellness, the present invention discloses a method and system for patient temperature monitoring and forecasting. Specifically, the method incorporate a deep learning framework in a HIPPA complaint data workflow to determine a stringent baseline core temperature, infer the onset of hypothermia and hypothermia from this baseline, and provide an option to rapidly assist nurses in patient thermoregulation using low-voltage sensors applied to the patient. The method employs the computational power of neural networks that are capable of computing combinations of a large number of variables. In doing so, it provides the foundations for more statistically significant correlation between parameters stored in Electronic Health Record datasets, patient demographics, and environmental variables in real-time.

Central thermoregulation approaches can be improved by enhancing the efficiency and precision of thermal energy delivery. One method to achieve this is by incorporating adaptive, real-time feedback systems using temperature sensors combined to patient attributes. These sensors could monitor the patient's skin and produce core temperature estimates more frequently, enabling the system to adjust heat levels. By integrating machine learning or deep learning algorithms, the method can predict optimal heating patterns based on patient data, adjusting heat delivery to maintain a stable core temperature even as conditions change during surgery. This real-time adjustment minimizes energy waste and reduce the likelihood of temperature fluctuations.

Another application for the present method lies in the design of the compression system for blood flow regulation. Current intermittent compression methods can be augmented with more sophisticated, programmable pressure patterns tailored to each patient's circulatory system. Incorporating sensors to measure blood flow velocity and oxygenation levels in real-time allows for more targeted compression, improving venous return and the body's thermoregulation. Additionally, utilizing materials with higher flexibility and breathability in the compression sleeves enhances patient comfort, especially during longer surgical procedures.

Additionally, the present invention lends itself to a more portable and easier to use system in comparison to the prior art. Home-based, wearable, stylish and comfortable temperature trackers provide healthcare professionals the tools needed to analyze unique individual temperature profiles. This can be accomplished by shrinking the size of the central unit, and integrating advanced battery technology or wireless energy transfer methods to allow greater mobility and ease of setup. Thus, the entire system is redesigned with modular, flexible components, allowing medical staff to apply and remove bulky parts quickly and efficiently. Additionally, integrating this technology into wearable devices that provide continuous monitoring and regulation of body temperature beyond the operating room makes it a more versatile tool for patient care, extending its use to pre- and postoperative settings as well.

These and other advantages of the present invention will best be appreciated by reference to the Detailed Description Of the Preferred Embodiments below in conjunction with the accompanying drawings listed below.

### BRIEF DESCRIPTION OF THE DRAWINGS

FIG. 1 illustrates a patient bay setup along with the physical arrangement of additional monitoring devices, charging stations, and data synchronization equipment;

FIG. 2 illustrates a flexible thermoelectric cooler assembly, including the thermoelectric module and flexible insulators;

FIG. 3 illustrates a schematic of a custom-printed circuit board for the thermoregulation system;

FIG. 4 illustrates front and back views of the data collecting bands, including sections to embed the thermoelectric cooler, battery and a printed circuit board;

FIG. 5 illustrates the physical anatomical placement of the bands of FIG. 4;

FIG. 6 is a flow chart of the integration pathway for healthcare systems;

FIGS. 7A-D is an overview of an ensemble recurrent neural network to produce core temperature estimates and core temperature predictions;

FIGS. 8A, B is a flow chart illustrating the assist mode operation, showing the activation process and thermoregulation feedback loop; and

FIG. 9 is a schematic diagram illustrating the interaction between hardware, software, cloud services, and data flow for core temperature estimates, prediction, and management.

### DETAILED DESCRIPTION OF THE PREFERRED EMBODIMENTS

The following are definitions of terms used in this application.

Bands, or “SITIS” bands, refers to an electronic device used for the acquisition of physiological data and the regulation of core body temperature via the skin. The band monitors breathing rate and heart rate, providing data to a deep learning framework integrated with Electronic Health Records in the cloud, which generates core temperature estimates and predictions.

Flexible Thermoelectric Cooler refers to a compact, bendable thermoelectric cooling module that uses the peltier effect to transfer heat from one side of the module to the other when a DC current is applied. The device consists of thermoelectric semiconductor materials arranged in a series of thermocouples on a flexible substrate, to allow it to conform to the shape of the body or wearable device.

Flexible PCM refers to a layer of thermoregulation material that undergoes a reversible phase transition between solid and liquid states to absorb or release heat, providing passive thermal management. The material's phase change must occur at a specific temperature range.

Secondary Contact Plate refers to a flexible component layer to enhance thermal conduction between a thermoelectric cooler and the skin. It acts as an intermediary layer that ensures uniform heat distribution by making consistent contact with a flexible thermoelectric cooler.

Flexible Printed Circuit Board refers to a thin, lightweight, and highly flexible substrate used to connect electronic components in a compact and durable manner. The Flexible Printed Circuit Board is constructed using polyimide or polyester materials and incorporates multiple layers to route signals efficiently while maintaining flexibility and durability, even under continuous motion.

WI-Fi Module refers to a WiFi and Bluetooth 6 Microprocessor with an on-board antenna.

Respiration Sensor I/O refers to the analog input/output pins or ports on a microcontroller or integrated circuit that interface with the electrical signals generated by a respiration sensor. These signals, resulting from mechanical deformations during inhalation and exhalation, are sent through the I/O pins to an Analog-to-Digital Converter for digitization and subsequent processing by the microcontroller.

Thermistor #1 refers to a temperature dependent resistor.

Clock Management IC refers to a two pin, quartz ceramic resonator integrated circuit.

Heart Rate Sensor I/O refers to the analog input/output pins or ports on a microcontroller or integrated circuit that facilitate the connection and processing of electrical signals generated by the heart rate sensor. These pins must handle the transmission of continuous, variable signals from the sensor for processing or output calibrated signals to other components in the circuit.

Power Management IC refers to a USB-friendly lithium-ion battery charger and power-path management integrated circuit.

Voltage Regulator refers to a miniature step-up boost voltage regulator utilizing a 1.6 MHz frequency.

Data Security IC refers to a cryptographic security AES SP.

Ambient Temperature & Humidity Sensor refers to a low power, high accuracy digital humidity sensor with a temperature sensor module.

Flash Memory IC refers to a 8-Mbit, 2.7 to 3.6V, SPI flash memory integrated circuit.

Battery Charge IC refers to a USB-friendly lithium-ion battery charger and power-path management integrated circuit.

Battery Surge IC refers to a UV, OV and reverse supply protection controller equipped with a 50/60 Hz noise rejection and an overvoltage/undervoltage reverse-polarity protection.

Driver for Thermoelectric Cooler refers to a power driver for a peltier TEC module, rated at +−3 A.

ESD Protection refers to a quad bidirectional transil, suppressor for ESD protection, with a 5V standoff and with 4 Channels.

Buzzer refers to a polarized quartz resonator, ceramic.

Back Up Relay refers to a 5V solid-state relay designed for high switching speed.

Peripherals refer to a 4-channel I2C or SPI multiplexer for efficient connection of the heart rate sensor, respiratory sensor and ambient temperature sensors.

Nordic Semiconductor refers to a family of ultra-low-power System-on-Chip (SoC) solutions equipped with Bluetooth 5.0 radios, ARM Cortex-M processors, and integrated power management, optimized for wireless communication in IoT applications. The SoCs must provide low-latency data transmission between wearable sensors and an IoT edge device. The SoC needs to operate with a 1.5 to 3.6V supply voltage.

Heart Rate Data refers to a time-series physiological signal representing the electrical activity of the heart, derived from photoplethysmography or electrocardiography. The signal must be acquired at high-frequency sampling rates from 125-1000 Hz, generating continuous waveform data that is subject to preprocessing algorithms such as signal detrending, bandpass filtering, and motion artifact removal. The resultant data is characterized by pulse peaks, from which instantaneous heart rate (beats per minute, BPM) is extracted using Pan-Tompkins or wavelet transforms. Raw heart rate data must be stored in floating-point format with a 32-bit precision.

Respiratory Rate Data refers to a continuous waveform that represents thoracic and abdominal movements associated with the respiratory cycle that is acquired through piezoelectric sensors. The raw signal is first subjected to bandpass filtering to remove noise from unrelated body movements. Respiration cycles (inhalation/exhalation) are identified using zero-crossing detection or signal envelope extraction, from which the respiratory rate (breaths per minute, BPM) is computed. Respiratory rate data is stored as floating-point vectors.

Skin Temperature Data refers to the raw analog signals representing temperature measurements, ranging from 0 to 100 degrees Celsius. These signals are converted into digital form using a 12- to 16 -bit analog-to-digital converter to achieve higher resolution and accuracy.

Environmental Data refers to temperature and humidity data. The environmental temperature is logged with ±0.1° C. accuracy using thermistors or thermocouples, while humidity is measured via capacitive humidity sensors with a resolution of ±1% RH. Environmental data is archived as a time-stamped record in a multidimensional array.

Electronic Health Record Data refers to structured data integrated via Health Level 7 or Fast Healthcare Interoperability Resources APIs, retrieved from a secure cloud database. This structured data includes patient demographics, historical temperature readings, medication history, laboratory results, and physiological disorders. EHR data is encoded in a hierarchical structure, with critical variables of height, weight, medication, age, race, sex, and the time of the day extracted and preprocessed for input into the deep learning model. The EHR data is stored in encrypted formats and is processed within cloud-based environments, on-premises environments or hybrid cloud environments.

Mean Absolute Error refers to a statistical metric used to evaluate the performance of a predictive deep learning model. The software computes the mean absolute error by taking the absolute difference between predicted and actual core body temperatures, averaged across all samples over a defined time horizon.

Kalman Filter refers to a recursive algorithm used to estimate the state of a dynamic system by minimizing the variance between predicted and measured values, in noisy biological data. The Kalman Filter processes heart rate, respiratory rate and skin temperature data. It must operate by executing two core steps: prediction and update. In the prediction phase, the filter estimates the next system state based on a previous state and a first-order Markov process. In the update phase, new measurements are incorporated, and the system's state is corrected using weighted averages, where weights are determined by covariance of the prediction and measurement. The filter continuously recalibrates its estimations by blending past information with new sensor data.

LSTM 2 refers to a long short-term memory model that must be trained on proxy indicators for blood perfusion rates such as body temperature, heart rate, systolic blood pressure, diastolic blood pressure, mean arterial pressure, lactate, oxygen saturation, and respiration rate from Electronic Health Record data. It uses a sigmoid activation function.

Markov Blanket refers to a probabilistic technique that isolates core body temperature-relevant variables, including vasodilation/vasoconstriction probabilities, heat flux, peripheral temperature, heart rate, and respiratory rate. It must operate by establishing a boundary of conditionally dependent variables to minimize noise and reduce uncertainty during training and deployment.

LSTM 1 refers to a long short-term memory model that must be trained for a correlation task using time-series data such as such heart rate, systolic blood pressure, diastolic blood pressure, respiratory rate, oxygen saturation, body temperature, and mean arterial pressure from Electronic Health Record data. It uses a softmax activation function.

Binned Markov Blanket refers to a probabilistic technique of identifying the conditional dependencies and independencies in a model by grouping variables into discrete categories. It must operate by segmenting continuous variables, such as heart rate, skin temperature, and ambient conditions, into bins or ranges that represent distinct states.

LSTM 3 refers to a third long short-term memory model that must be trained on time-series data using only blood perfusion and body temperature data from Electronic Health Record data. It uses a softmax or linear activation function.

Dense Output Layer 2 refers to the final layer of an ensemble recurrent neural network producing the predicted core body temperature as a single continuous value. This layer must employ a linear activation function.

Final Output Neuron refers to the last neuron in an ensemble recurrent neural network that produces a core body temperature prediction.

Data Lake refers to a centralized repository on the cloud to store raw, unstructured, and structured output results from an ensemble recurrent neural network.

Monte Carlo Quantiles refers to a statistical method to estimate the aleatoric and epistemic uncertainty in core body temperature predictions.

Event-Driven Function refers to a self-contained code module designed to execute in direct response to predefined hardware or software triggers. These triggers need to include peripheral sensor data changes, hardware interrupts, or system-level software events. The function needs to operate within an asynchronous execution model and must be designed to handle specific event payloads or state transitions while maintaining compatibility with the broader system architecture.

Dubois Equation refers to a mathematical formula to calculate the Body Surface Area, a key parameter for estimating heat flux and metabolic rates. The equation uses weight, measured in kilograms, and height, measured in centimeters, to derive an individualized BSA value.

Body Surface Area Calculation refers to the method of estimating the total surface area of a patient's body, which is derived using the Dubois equation. This calculation considers weight, measured in kilograms and height, measured in centimeters to provide an accurate measure of body surface area.

Pennes Bioheat Equation refers to a mathematical model modified with heterogeneous blood perfusion that simulates heat transfer and temperature distribution within biological tissues. It uses tissue temperature, estimated from filtered thermistor readings of skin surface temperature, with constants being thermal diffusivity, tissue density, specific heat, and tissue thermal conductivity, while blood perfusion rate and blood temperature are derived from patient records. The modified heat flux equation introduces an evaporative heat loss component influenced by humidity, calculated using the evaporation rate from the skin's surface and body surface area, determined through the Dubois equation. Additionally, the equation considers the convective heat transfer coefficient and ambient temperature for precise heat exchange predictions.

Heat Flux Values refer to the rate of heat transfer per unit area of skin and is calculated based on metabolic heat generation, tissue thermal conductivity, and blood perfusion using Pennes Bioheat Equation. These values need to quantify the amount of heat exchanged between the skin tissue and the environment.

EMD refers to an Empirical Mode Decomposition, which is a signal processing technique to decompose nonlinear heat flux value into intrinsic mode functions. This method needs to allow for the extraction of oscillatory components from the time-series data.

IMFs refer to Intrinsic Mode Functions, which are the oscillatory components extracted from heat flux values using an empirical mode decomposition technique.

Blood Perfusion Value refers to the rate at which blood flows through the microvasculature of the human body.

Blood Temperature Value refers to the temperature of the blood circulating through the human body.

Patient Attribute Data refers to non-time-series demographic data such as age, sex, weight, height, race, ethnicity and time of day.

Oxygen Saturation refers to the percentage of hemoglobin in the blood that is bound with oxygen.

Mean Arterial Pressure refers to the average pressure in the arteries throughout one complete cardiac cycle, encompassing both systolic and diastolic pressures.

Golden Vital Signs Weights refer to a section of code that assigns importance to vital sign inputs.

Golden Temperature Threshold refers to a section of code in a data preprocessing step to specify the standard and clinically relevant range for core body temperature.

Optimal Temperature Value refers to a number representing the inference temperature point. It is measured to the hundredths place.

Assist Mode refers to an optional feature of the present invention that allows healthcare professionals or a consumer to manage body temperature with autonomous heating or cooling support to the integumentary system.

Trends refer to a module in the frontend using a symbol to denote heating or cooling states for a given user or patient.

Settings refers to the functionality of allowing the user to update firmware, access customer support, manuals, certifications, and terms and conditions.

Battery Status refers to a dedicated section of the display to monitor battery status, estimated time remaining, and notifications for recharging.

User Guidance refers to in-app or external guidance, tutorials and FAQs to help users understand how to effectively use the device and app, manage settings, and interpret data.

Historical Core Temperatures refer to the previously recorded core body temperature values over time.

Manual Mode refers to the user-initiated adjustments to the thermoregulation system designed to modify the temperature of the body with user-defined duration values for heating or cooling on specific body sites.

User Duration Value refers to the length of time a SITIS band has been set to in order to maintain or adjust core body temperature.

Peltier Duration Value refers to the time the Peltier thermoelectric cooler is on.

Heart Rate Sensor refers to an electro-optical device that employs photoplethysmography to measure the volumetric change of blood in microvascular tissues. It operates using an infrared light source and a photodetector, capturing variations in light absorption corresponding to pulsatile blood flow. The sensor has a dynamic range capable of measuring heart rates from 30 to 240 beats per minute with a precision of ±2 bpm. The sensor operates on 3.3 volts. This data is converted into electrical signals, which are then processed by the Analog-to-Digital Converter before being transmitted to the MCU.

Respiration Sensor refers to a device that uses piezoelectric materials to measure the expansion and contraction of the chest or diaphragm. The sensor captures mechanical deformations caused by inhalation and exhalation. These variations are converted into an electrical signal, which are sent through the system's ADC to be digitized and processed by the MCU.

Thermistor refers to a temperature-sensitive resistor constructed from semiconductor materials, with a high negative temperature coefficient.

ADC refers to an analog to digital converter which is a dedicated integrated circuit responsible for converting continuous analog sensor signals into discrete digital signals that can be interpreted by the MCU. The ADC must operate at high precision, either a 12-bit or 16-bit resolution depending. It must interface with the sensors via direct wiring, using high-impedance inputs to accurately capture low-voltage analog signals. The converted digital signal must then be transmitted to the MCU through standard communication protocols, such as SPI or I2C.

Ambient Temperature and Humidity Sensor refers to a digital or hybrid module that combines a capacitive humidity sensor with a thermistor to measure both the environmental humidity and ambient temperature. The capacitive humidity sensor must consist of a hygroscopic dielectric layer sandwiched between two conductive electrodes. As humidity levels change, the dielectric constant varies, altering the capacitance, which is then converted into a digital signal. The integrated thermistor needs to measure ambient temperature, and both signals are sent to the MCU.

MCU refers to a microcontroller which is a small, embedded computing device that serves as the central processing unit. It is responsible for gathering, processing, and transmitting data from sensors such as heart rate, respiratory rate, and skin temperature to an IoT edge device.

Electronic Health Record refers to digitized medical records maintained by healthcare institutions, which utilize clinical datasets structured in databases or other formats designed for efficient data storage and retrieval.

Cloud-based IoT Software Development Kit refers to a collection of libraries and APIs that facilitate communication between MCU and a cloud infrastructure. The SDK should include data architectures for MQTT, AMQP, or HTTP/2 protocols and enables JSON and Protocol Buffers. It should handle device provisioning, message encryption, and device-to-cloud data transmission. The SDK should also include device over-the-air management and secure boot procedure.

Cloud-based IoT Hub refers to a centralized platform that facilitates communication and data management for MCU and IoT edge devices.

Iot Edge Device (Ubuntu) refers to an edge computing platform that serves as an intermediary layer between sensor wearables and an IoT Hub.

Cloud-based IoT Connector for FHIR refers to a middleware program that enables secure, standardized exchange of healthcare data between IoT edge devices and Electronic Health Record systems. The connector must implement FHIR-compliant RESTful APIs to allow structured physiological data and be seamlessly integrated into clinical workflows. It must handle the mapping of sensor data into FHIR resource types and ensure data exchange follows healthcare standards, including encryption (AES-256) and authentication (OAuth 2.0).

Kubernetes Cluster refers to a software architecture that orchestrates the deployment, scaling, and management of containerized microservices that make up the horizontally scalable ensemble recurrent neural network. This infrastructure needs to automate the process of container scheduling, load balancing, and failover. It needs to provide built-in security features such as role-based access control, network policies, and encrypted storage to ensure compliance with HIPPA policies.

Pod #1 refers to the smallest deployable unit in a Kubernetes ecosystem consisting of one container running a specific application. This pod must operate in a stateless manner and be designed to be horizontally scalable. Each pod needs to be associated with a Persistent Volume Claim to store intermediate results or logs and interact with an ensemble recurrent neural network through an API.

Image refers to a packaged and immutable file containing the entire environment needed to run an application including the code, runtime libraries, and dependencies. The image needs to include a containerized Python environment with TensorFlow or PyTorch pre-installed, along with Flask for API endpoints and Pandas for data manipulation. This image also needs to host a pre-trained ensemble recurrent neural network.

API for FHIR refers to Fast Healthcare Interoperability Resource, which is a secure standardized program to interact with healthcare systems and retrieve patient data in a structured format. This API needs to use RESTful web services that conform to the FHIR specification, enabling the thermoregulation system to access, update, and store health-related data. The API needs to support both synchronous and asynchronous communication modes.

Core Temperature Prediction Engine refers to an ensemble recurrent neural network trained specifically to both estimate and predict core temperature based on heart rate, breathing rate, skin temperature, environmental temperature and Electronic Health Record data. It must use three long short-term memory models built in TensorFlow or PyTorch and trained on Electronic Health Record datasets.

ML Workspace refers to a dedicated software environment for developing, training, and deploying machine learning models. The ML Workspace must integrate the libraries and computational resources necessary for handling large-scale data processing, model training, and inference tasks. It needs to include support for TensorFlow, PyTorch, or scikit-learn, and be optimized for parallel computation using TPU, GPU or multi-core CPU resources.

Thermoelectric Cooler Plate Section refers to the surface in the front side of SITIS100 to seamlessly fit a thermoelectric cooler plate.

Outer Casing refers to the final outer layer of the SITIS band.

Thermoelectric Cooler Section refers to the machined section of the SITIS band designed specifically to house a flexible thermoelectric cooler.

PCB Slot refers to a machined portion of the hardware designed to house a custom printed circuit board.

Battery Slot refers to a machined portion of the SITIS band designed to house a battery and circuit.

Straps refer to a hook-and-loop flexible material to secure SITIS bands around the body.

RGB Indicator Light refers to an RGB attached to the SITIS band.

Dome Switch refers to a tactile, pressure-sensitive switch attached to the SITIS band.

Skin Temperature Value refers to the temperature of the skin.

Temperature Matching refers to a software step that is used to align the incoming vectors of skin temperature to current vectors of skin temperature.

Float Temperature Value refers to a temperature measurement represented as a floating-point number format.

Calculated Error refers to the difference between the desired temperature setpoint and the actual measured temperature, which is used to assess the performance of the thermoregulation system. The error is continuously computed as the absolute or relative deviation between the predicted or target core and skin temperatures and the real-time data gathered from the sensors. This error value serves as a critical input to the PID controller and other control algorithms, driving the activation or modulation of a thermoelectric cooler.

PID Controller refers to the control mechanism within the thermoregulation system that uses proportional, integral, and derivative terms to adjust and maintain the patient's core and skin temperature within a desired range. The controller continuously evaluates the error between the desired temperature setpoint and the actual measured skin temperature from sensors. The proportional term computes a correction that is directly proportional to the current temperature error, adjusting the thermal output to bring the temperature closer to the setpoint. The integral term addresses accumulated past errors over time, compensating for any drift or offset in the system to ensure long-term accuracy. The derivative term predicts future temperature changes by assessing the rate of change in the error, allowing the system to preemptively adjust and avoid overshooting or oscillations in skin temperature control.

Peltier Duration and Direction Correction refers to a software step to adjust the duration and direction of the Peltier thermoelectric module's operation to maintain the patient's thermal homeostasis. Based on a PID controller, the system dynamically corrects the heating or cooling duration and direction to stay within the target skin temperature range.

Peltier Activation refers to engaging the Peltier thermoelectric module to heat or cool the patient's skin based on the core temperature-skin temperature correlation The module needs to apply thermal flux or remove thermal flux to regulate the patient's skin temperature.

Request Core Temperature Estimate refers to the process of obtaining an estimated core body temperature based on real-time sensor data and a predictive model. This request triggers a core temperature prediction engine to process the data using deep learning methods, such as LSTM networks, to provide an immediate core temperature estimate.

Optimal Skin Temperature Value refers to skin temperature values that are found to best help maintain core body temperature homeostasis for a given window of time during the thermoregulation assistance of the integumentary system.

Notifications refer to system-generated alerts sent to users or administrators about critical updates, warnings, or status changes in the thermoregulation system. These should be triggered by events like changes in core temperature, abnormal sensor readings, system anomalies, and are delivered through channels such as apps, email, and messaging from a backend server.

Sensor Calibration refers to the software step of adjusting and fine-tuning sensors to ensure accurate and reliable readings in the thermoregulation system. It needs to involve comparing sensor outputs to known references and applying corrections for offset, gain, and linearity. Calibration must be performed periodically to account for sensor drift.

Read Sensor Data refers to the process of capturing real-time measurements from physiological sensors through the ADC in the MCU. It must involve initializing sensor interfaces, reading raw values, converting them into standardized units, and storing the data for processing. The data should then be transmitted securely to an IoT hub for further analysis.

Transmit Sensor Data refers to the process of securely sending sensor readings via integrated bluetooth modules to a centralized MCU, and from a single centralized MCU to an Iot Edge Device via an integrated Wi-Fi module. It involves aggregating and calibrating sensor data, formatting it into standardized packets, and using secure communication protocols for transmission.

System Check refers to the process of verifying the operational status and integrity of the thermoregulation system, including the IoT edge device and sensors. It ensures proper device power, sensor functionality, and stable communication for data transmission. The process also involves validating the machine learning model and troubleshooting any data flow or hardware issues.

ASSIST MODE Check refers to an algorithmic validation process inside an MCU to ensure the proper functioning of the assistive thermal regulation mode. It verifies input from the frontend.

ASSIST MODE ON refers to the functionality of an MCU to use a thermoelectric cooler to regulate core body temperature based on determined optimal skin temperatures by the SITIS100 software.

Heating refers to a controlled process in which thermal energy is applied to elevate the skin temperature.

Cooling refers to a controlled process using thermoelectric coolers, where the cold side of the device removes thermal energy to lower the skin temperature.

New Skin Temperature refers to the real-time measurement and processing of skin surface temperature using wearable sensors, which digitize and preprocess the data for accuracy. The cleaned data is then securely transmitted via encrypted protocols to a cloud-based platform for integration into the thermoregulation system.

New Ambient Temperature refers to the measurement of the surrounding environmental temperature using an ambient temperature sensor.

Heat Flux Calculation refers to the process of quantifying the rate of heat transfer from the body to the environment. The calculation is derived from the Pennes Bioheat Equation.

Optimal Skin Temperatures refer to the collective skin temperatures that have been determined by the SITIS100 software to best support core-temperature homeostasis.

Mobile App Display refers to the frontend display in the mobile application for the SITIS100 thermoregulation system.

Data Lake refers to a centralized repository that stores vast amounts of structured and unstructured data collected from various sources within the thermoregulation system.

ASSIST MODE OFF refers to the state in which a thermoregulation system operates without any active interventions. In this mode, the system ceases to automatically adjust thermoelectric cooler duration and direction.

System Alert refers to an automated notification or warning generated by a buzzer to signal abnormal conditions or potential failures in the system's operation. This alert is triggered based on predefined thresholds, such as discrepancies or malfunction in sensor data irregularities, or errors in thermoelectric coolers.

System Shutdown refers to the controlled deactivation of the thermoregulation system, initiated automatically in response to critical operational conditions or external factors that necessitate the cessation of system functions. During a system shutdown, all active processes, including data collection, processing, and actuation of heating or cooling devices are halted. The system stops receiving or transmitting sensor data, and all system functionalities are paused until a restart or manual intervention is performed to restore operations.

Power refers to the initial activation state of the thermoregulation system, where electrical power is supplied to the sensors, processors. This event triggers the initialization of the microcontroller unit and its associated peripherals, such as the heart rate, breathing rate, and skin temperature sensors. Upon power-on, the system begins to collect sensor data and initialize the data pipeline for the core temperature prediction engine. Upon power-off, the microcontroller unit halts data collection and all operations are properly terminated.

Esophageal temperature monitoring technology provides highly accurate, continuous core temperature monitoring by inserting a probe to a depth of approximately 30 to 40 cm into the esophagus, positioning it near the heart and major blood vessels, where temperature closely reflects the core body temperature. The system relies on thermistor-based sensors composed of manganese, nickel, or cobalt oxides, which exhibit predictable changes in electrical resistance in response to small temperature variations. The probes themselves are constructed from biocompatible materials, such as medical-grade silicone or polyurethane, designed to minimize irritation and discomfort during prolonged use. These enable safe and extended insertion periods of time inserted deep into the esophagus, a critical feature for continuous monitoring of the core temperature. The key data sensor relies on the thermistor within the probe to transmit real-time temperature data to external patient monitoring systems, where the signals are amplified, digitized, and integrated into a hospital's centralized monitoring platform. This platform then can consolidate data from multiple physiological parameters, providing clinicians with a comprehensive view of the patient's vital signs.

Another key method in core temperature monitoring is zero-heat-flux (ZHF) thermometry, a non-invasive technology that offers high-precision core temperature measurements without requiring internal probes. The system operates on the principle of thermal equilibrium, aiming to eliminate the heat exchange between the sensor and the skin by applying thermal insulation and active heating elements. The ZHF system consists of a dual-sensor array, one sensor placed on the skin to measure temperature and another sensor to measure heat flux. This setup nullifies the thermal gradient between the skin and the core by actively heating the skin to match the body's core temperature. This method is especially advantageous for patients requiring long-term monitoring where invasive methods pose risks. The engineering foundation of ZHF technology is based on thermodynamic principles, wherein the heat flux sensor detects the rate of heat transfer between the skin and the environment. Embedded heating elements can then continuously adjust the skin surface temperature, preventing heat loss or gain from the surroundings. As heat flux approaches zero, the temperature at the sensor matches the body's core temperature.

Bladder thermistor catheter technology represents another critical approach to core body temperature monitoring in hospital settings. This system functions by using the temperature of urine within the bladder, which correlates closely with core body temperature due to the bladder's proximity to large blood vessels and internal organs. The catheter is designed to integrate a thermistor sensor into the tip of a foley catheter, a device normally used for urinary drainage. The bladder thermistor catheter operates by embedding the thermistor into the catheter tip, which remains in direct contact with the urine as it fills the bladder. Given that urine rapidly equilibrates to the core body temperature through heat exchange with surrounding tissues, the thermistor can then detect minute temperature changes with high sensitivity. The detected temperature is converted into an electrical signal that travels along a conductive wire embedded within the catheter to an external monitoring unit. The system's signal processing electronics interpret these electrical signals and translate them into precise temperature readings within a ±0.1° C. accuracy.

Microcontrollers in modern thermoregulation systems that run core temperature estimation algorithms often implement classical computational models such as linear regression or Kalman filters to derive core body temperature from peripheral measurements. The processing workflow involves data preprocessing, such as noise reduction through low-pass filters, followed by algorithms that map peripheral temperature signals to core temperature estimates. These classical algorithms incorporate signal processing techniques that consider heat transfer dynamics and thermodynamic principles. Once core temperature is inferred, the system's feedback control mechanism is activated, whether fluid or fan-based. This allows the microcontroller to act as the control unit within a closed-loop control system, sending control signals to actuators like high-voltage Peltier heating elements, resistive heating films, or mechanical fans via Pulse Width Modulation (PWM) or other digital I/O interfaces. The actuators are embedded in fabrics or used in combination with liquid elements to adjust the thermal output based on the difference between the inferred core temperature and the desired set point as set by a healthcare professional.

System architecture is further enhanced by the inclusion of real-time operating systems (RTOS), which ensure deterministic timing for temperature data sampling, processing, and control actions. Data flows continuously through the system in a real-time loop to send commands to actuators. The system periodically updates based on new data inputs, maintaining a steady stream of sensor input, processing, and actuator control. In terms of user interaction, these systems feature a Graphical User Interface (GUI) or a Human-Machine Interface (HMI), allowing users to manually set temperature thresholds or view real-time temperature data. The user interface communicates with the microcontroller via serial communication protocols such as UART, I2C, or SPI, enabling the manual input of temperature settings that override automatic control, if necessary.

The method and system of the present invention centers around a non-invasive, wearable core-body temperature monitoring, prediction and controlling system that integrates Electronic Health Record data into a deep learning framework coupled with low-voltage thermoelectric coolers selectively placed on focal thermoregulation skin. To be able to monitor, predict, and control core body temperature it is essential to understand the role of the integumentary systems in core temperature regulation. This understanding can be bridged by using low-voltage microcontroller units on major skin sites that assist with thermoregulation. This keeps in mind vasoconstriction and vasodilation during normal homeostasis processes.

The invention integrates deep learning software with Electronic Health Records and patient data attributes to further understand these physiological effects, determine optimal core temperature values, and non-invasively predict core temperature using changes of core temperature in an ensemble recurrent neural network (RNN) consisting of three long short-term memory (LSTM) models.

The present invention uses an ensemble recurrent neural network to generate a core temperature prediction. The method uses heart rate sensor data, respiration sensor data, and individual patient attribute data from an Electronic Health Record to be enriched in a HIPPA compliant API. It can then produce an inference between 30 seconds and 2 hours into the future to detect an early onset of hypothermia or hyperthermia.

Peripheral temperature thermoregulation analysis plays a key role in the process. The computational analysis of thermodynamic heat flux at multiple anatomical sites establishes the software architecture for modeling the dynamic equilibrium between peripheral and core temperature. This is achieved by continuously tracking spatiotemporal variations in the thermal gradient across the integumentary system as a partitioned entity of negative and positive heat flux.

To obtain the proper measurements, the method preferably employs wearable low-voltage (e.g., 5 volts) flexible thermoelectric coolers selectively placed on seven regions on the integumentary system. These coolers are placed adjacently to increase cooling and heating efficiency as needed. The use of low-voltage electronics concedes a low-humidity, non-liquid based thermoregulation method via the skin.

The thermoregulation methodology of the present invention operates through the integration of both hardware and software components. The core functionality of its software depends on training an ensemble RNN model with >350 million EHR data points accumulated over a 10-year period from over 600,000 unique patients using an RTX 4060 computer. In training, the ensemble recurrent neural network consists of 3 long short-term memory models, each with 3 layers to extract features from the following time-series EHR data: vital sign, laboratory, basic procedural, medication, assessment, surgical procedural, and diagnostic coding variables. In addition, a dense layer is used to extract features from the nontime-series patient attributes of weight, height, age, sex, race, and time of day. A dense hidden layer extracts additional features based on the output features from the 3 input LSTM layers. Each LSTM model learns to extract three 256-length vectors as intermediate features from the input, while the single dense input layer takes age, sex, race, and the time of day to extract a 16-length vector as an intermediate feature. The hidden layers containing these 3 vectors flow to a hidden layer consisting of 16 neurons plus a bias neuron. This final dense hidden layer extracts features from all the preceding inputs and presents a 16-length vector to the final output neuron that performs the actual classification. All uncertainties during training and deployment are quantified using Monte Carlo Quantiles in the backend using a cloud-based machine learning workspace.

In deployment, the trained and validated ensemble recurrent neural network (RNN) is serialized as a pickle file and then pushed into a cloud-based model registry using a proprietary machine learning workspace. The model, along with all its dependencies, including all equation calculations and the API for FHIR, are packaged into a container and deployed to a cloud-based Kubernetes cluster as a compute target. An IoT hub is used to facilitate bidirectional communication between an IoT edge device (Ubuntu) and a cloud-based Kubernetes cluster using a MQTT protocol. A filter module is used in the IoT edge device to send skin temperature values, heart rate and respiratory rate data to the cloud through an IoT Hub and up to a FHIR-compliant API, where the data is pre-processed.

The FHIR-compliant API is containerized within the same docker image as the ensemble RNN and is deployed in a Kubernetes cluster, functioning as a RESTful endpoint for exchanging clinical data in FHIR format. This enables the ensemble RNN to then be accessed for generating inferences, with its outputs sent to the FHIR API for storage in a data lake and retrieved by a cloud-based data factory for retraining. The prediction results are transmitted to both a mobile application and a web-based graphical user interface (GUI) by offloading the inferences from the container as JSON files to a backend server. This server processes the data for historical analysis and anatomical visualization.

Moreover, the static data input needed for the ensemble RNN is acquired from the institution's EHR system and formatted to connect to an api via a web service compliant with FHIR. The continuous data inputs needed for the ensemble RNN are breathing rate data, heart rate data, humidity data, and ambient temperature data. Sensor-based data is obtained using MQTT protocols, while EHR data undergoes FHIR mapping to normalize data, create a template, and produce FHIR data in the container. Registered IoT devices are preferred in a proprietary IoT HUB, but any cloud-based IoT service can be used.

The nature of anonymizing patient data involves a specificity in the predictive capabilities of the model. For this reason, a Kubernetes cluster is the preferred infrastructure to manage the data horizontally coming from each deployment; it includes the use of pods to manage container images and prevent any mishap on functioning based on any given computer specifications. In addition, to protect patient data, it is essential to implement data security protocols such as X509 and BAC to manage permission in registered IoT devices. Lastly, the choice to utilize a cloud based IoT hub to handle versioning, automatic scaling and monitoring of model performance is in tandem with the growing trust from healthcare institutions. These software foundations are aimed for the longevity of our ensemble RNN.

The predictive capabilities for individual core temperature begins in the anatomical placement of the SITIS bands. These bands serve as the 30-second telemetry route required to determine the heat flux of major thermoregulatory blood vessels during core body homeostasis. The sensor data and EHR data are processed in hardware and in the cloud respectively to be used for an IoT edge device, Ubuntu. The IoT edge device is deployed within a Kubernetes pod to enable remote operation and it is equipped with adequate memory and computational power to calculate heat flux values using Pennes'Bioheat Equation. This calculation aggregates evaporation from the skin surface area, which is determined using the body surface area (BSA) calculated through the Dubois equation and the evaporation heat flux equation.

Tissue conductivity is predefined as a constant value, while tissue temperature (T) is estimated from filtered thermistor readings representing the skin surface temperature (Ts). Skin blood perfusion and blood temperature (Tb) are requested from the patient record in an ICU facility and transmitted as Protected Health Information (PHIs). In addition, to align with HIPAA's data minimization principle, once a heat flux value is generated, PHIs are shredded from the IoT edge device. If unavailable, their constant value in a steady-state Pennes'Bioheat Equation is used. Seven heat flux values are then calculated sequentially and are transmitted to the API for FHIR for an EMD preprocessing step. This process involves applying Empirical Mode Decomposition (EMD) to partition the seven continuous heat flux values into distinct frequency components by decomposing the input time series into Intrinsic Mode Functions (IMFs). Each IMF captures oscillatory patterns within the heat flux data at different scales. By analyzing the individual IMFs, one can reconstruct and derive specific heat flux values corresponding to distinct heat loss and heat gain over time. Thus, the final use of the IMFs is to isolate these trends by differentiating high-frequency fluctuations from low-frequency components with respect to a reference temperature point and also compute new heat flux values by averaging the relevant IMFs in both negative and positive heat flux.

Next, to estimate core temperature a first LSTM model is trained on seven vital sign inputs from an EHR. Its function is programmed to perform a regression task on the acquired data and estimate a core body temperature based on seven vital sign features: heart rate, systolic blood pressure, diastolic blood pressure, respiratory rate, oxygen saturation (SpO2), body temperature, and mean arterial pressure (MAP). In training, this time-series data is engineered for three LSTM layers and captures the time-window differences between the data points structured in a 3-D array. This is performed by selecting custom timesteps to match the core temperature time granularity, engineering the features to fit the max and min values for the seven pertinent features, and applying the target core temperature in the last column for a dataset of more than 600,000 unique patients. This yields a core temperature estimate result that is evaluated with MAPE and RMSE to validate its accuracy.

The difference between estimating core temperature and creating a patient-centric core temperature threshold is defined by the addition of a custom layer containing predefined weights applied to the vital signs input, based on given importance values by healthcare professionals. This is followed by learnable optimal min and max values to penalize inferences made outside of an initial optimal temperature value range, representing a potentially normative temperature threshold of 95.3-100.4 degrees Fahrenheit. The gradients of the loss function with respect to the weights given can then enforce a learning curve within the first LSTM model to make subsequent inferences closer to a new optimal temperature value range. As the number of epochs grows larger, the distribution of core temperature inferences shifts towards the actual temperature range observed, representing non-bounded optimal min and max value created by a linear activation function. This process is aimed to provide a more accurate optimal temperature threshold representative of a patient's true hyperthermia and hypothermia values based on the correlation between the seven vital sign features aforementioned. Accuracy is measured in a MAPE metric and the deviation from the optimal value is measured in an RMSE metric. In deployment, a mean absolute error step is used in the IoT edge device for parameter recalibration in a Kalman filter using any of the available vital sign features. Lastly, the ambulatory consumer version of SITIS100 artificial intelligence-powered thermoregulation system uses only heart rate and breathing rate data to estimate the core body temperature in a lightweight model, while keeping all of its software steps.

Secondly, to create a core temperature prediction window for a given patient, it is necessary to understand vascular tone, specifically, the concurrent vasodilation and vasoconstriction states of its major thermoregulatory skin vessels. This process begins by first using only the estimated core temperature outputs of the first LSTM model as an input for a first Markov Blanket. The Markov Blanket provides the probabilistic context for core temperature in parent, children, and co-parent variables in a correlational relationship. This ensures that only those factors that have direct or indirect causal effects on core temperature can be passed to the second LSTM, effectively lessening noise for larger datasets overtime.

Furthermore, the output of this first Markov Blanket and the two IMFS created in the earlier EMD step are now eligible to be preprocessed for a second LSTM model. In the API, a small calculation is required to average out oscillatory trends of the IMFs to calculate new heat flux values, and subsequently determine new blood perfusion rate values. These rate values are used in the second LSTM model, which is trained on vascular tone indicators to infer vasodilation or vasoconstriction states. However, to successfully have the model learn vascular tone features, custom weights are added by healthcare professional to serve as proxy indicators for blood perfusion in the second model: core temperature, heart rate, systolic blood pressure, diastolic blood pressure, lactate, oxygen saturation (SpO2), and respiration rate, with the largest weight applied on lactate and the second largest on systolic blood pressure.

To ensure this is done correctly, the gradients of the loss function concerning the model's weights are utilized to optimize the learning curve, imposing penalties on classification inferences that fall outside systolic blood pressure values of less than 90 mmHg and greater than 120 mmHg, and lactate levels above 2 mmol/L; elevated lactate levels above 2 mmol/L are strongly correlated with states of vasoconstriction, as they often indicate impaired tissue perfusion and a shift to anaerobic metabolism due to reduced oxygen delivery.

Moreover, given the unique blood flow demands and the complex physiological responses of skin vessels, these penalties now serve as constrained proxy indicators, facilitating the second LSTM model to better predict blood perfusion. The result utilizes a sigmoid activation function and is directly interpreted as the state of vasodilation and vasoconstriction from blood perfusion rates under the SITIS bands, structured as a binary probability of 0 and 1. The outputs of the second LSTM now serve as inputs for a second binned Markov Blanket. This aims to enhance the predictive accuracy of the ensemble recurrent neural network by systematically selecting and binning the probability mass function of vascular tone.

Thirdly, the outputs of both the first LSTM model and the second binned Markov Blanket model are used as inputs for a third LSTM model. The binned Markov Blanket encapsulates critical information regarding vascular tone, specifically modeling the binary states of vasoconstriction (1) and vasodilation (0). These states are represented as a probability mass function (PMF), which quantifies the likelihood of each state occurring. By incorporating the binned Markov Blanket into a third LSTM model, the model's ability to capture long-term dependencies in sequential data is enhanced, particularly where core temperature events are influenced by prior blood perfusion proxies.

These proxies, represented probabilistically through the PMF derived from the binned Markov Blanket, are integrated as features into the third and final LSTM model of the ensemble RNN. The third LSTM is trained to target a core temperature based on the minimum and maximum systolic blood pressures within the Electronic Health Record dataset. In deployment, the moment of calculation for binary states and systolic blood pressure retrieval are attached to timestamps and are preprocessed to best align with the model's timesteps. This is perhaps the most difficult data preprocessing step and is programmed carefully to prevent data leakage and optimize learning. The resulting input layers are composed of the features most pertinent to a core temperature inference. The actual prediction occurs in the final output neuron using a softmax function, resulting in a model that is able to predict core temperature based on the binary state of vascular tones on seven thermoregulatory skin sites.

For a given offset prediction window, and based on empirical evidence of the strong causal relationship between blood perfusion and vascular tone, the thermoregulatory function of the integumentary system can now be reliably detected and modeled. The aforementioned processes in this system pipeline are created for real-time core temperature estimates and predictions based on the thermoregulation ability of the integumentary system through vascular tone as evidenced by blood perfusion trends during core temperature homeostasis. Collectively, the ensemble recurrent neural network forms the core temperature prediction engine.

To quantify the uncertainty in the ensemble RNN, the Monte Carlo dropout technique is employed. This method estimates both epistemic and aleatoric uncertainties by performing multiple forward passes, specifically 100, 100,000, and 1,000,000 iterations, each producing different outputs due to random dropout of model units. This approach approximates the posterior distribution of the model's predictions without the need for explicit Bayesian modeling. From this distribution, the method can extract the 5th, 50th, and 95th percentiles in each prediction window to capture potential deviations from expected core temperature. However, by selecting the 5th and 95th percentiles, we can define an acceptable 90% confidence interval that encompasses the range within which 90% of the predicted values fall.

Moreover, to quantify epistemic uncertainty, the predictions are averaged, and the variance across the stochastic realizations is computed, yielding the mean, or the expected value, and variance of predicted core body temperature and partitioned heat flux over time as continuous functions of the input data. To quantify aleatoric uncertainty, which encompasses the inherent noise present in the data, the model incorporates a variance term into its output during training. This variance term captures data uncertainty stemming from measurement errors and unpredictable fluctuations associated with the underlying physiological processes. By training the model to predict both the mean and variance of the output, aleatoric uncertainty is represented as the predicted variance across the outputs during each forward pass. This variance term accounts for the irreducible uncertainty that is naturally inherent in all physiological data.

During the optional “assist mode”, the training data is processed in sliding windows to detect time-series patterns of heat across a created thermistor sensor grid, or the hardware. The core functionality of the hardware in the system can be achieved through a custom printed circuit board (3.8 Volts) connected to low-voltage (5 Volts) thermoelectric modules, specifically flexible Peltier modules. These modules can then actively regulate body temperature by either generating heat or removing heat, depending on the analysis performed by the core temperature prediction engine. The engine recalculates new tissue temperature values based on the found optimal core temperature values, taken as blood a blood temperature value in Pennes'Bioheat Equation.

In addition, accelerating local heat exchange in specific regions, such as the head, chest, or extremities, can dramatically improve thermoregulation by enhancing heat dissipation or conservation where needed most. Therefore, to maintain an optimal core temperature, understanding the recurrent patterns of the body's natural thermoregulatory mechanisms are essential, particularly when managing sudden changes in temperature. The effects of these sudden changes can be inferred by the core temperature prediction engine to give a time interval where spikes in heat gain are likely to happen, specifically and as adjusted by a healthcare professional for a set core body temperature spike above a given threshold. With this approach, targeted heating or cooling in thermoregulatory skin tissue sites can then help to accelerate the body's natural ability to reach thermal equilibrium, supporting the body's vasodilation and vasoconstriction alongside other interventions in an ICU.

Due to the thermodynamics of the skin and the strain on a smaller thermoelectric cooler in combination to the heat dissipated by the MCU, the present invention intermittently warms and cools the patient based on humidity and ambient temperature data generated across each selection of skin. The system can then modulate the intervals and current direction of a set voltage applied to the thermoelectric coolers based on the real-time data and the inferences made, ensuring that core temperature regulation is assisted indirectly through the unique patient temperature regulation patterns. To power each of the thermoelectric coolers, a rechargeable battery coupled with a battery management integrated circuit can be manufactured to ensure efficient energy consumption and longevity to give an operating range of approximately 3 hours to 15 hours at a time. The design also incorporates a certified Wi-Fi module operating with an antenna, allowing for seamless data transmission to a HIPAA compliant server, where firmware can be updated over-the-air. Finally, before hyperthermia and hypothermia can be made evident and standardized for a given patient, it is pivotal to introduce a Monte Carlo or perturbation drop out to quantify uncertainty in the models'predictive abilities.

The following is a preferred set of steps to carry out the method of the present invention:

### Operating Procedure

1) Prepare the Skin
Clean, disinfect and dry the skin areas where the SITIS bands will be placed.
2) Place the SITIS Bands
Attach each SITIS band to the designated body region
3) Power on the SITIS Bands
Press and hold the button on the side of the SITIS band (carapace) to power it on
4) Activate the Designated Application on a Tablet.

Log in using your professional credentials. When prompted, enter the SITIS Number located on Band 3's carapace to establish a connection. Band 3 aggregates Bluetooth data from all the other bands and send it to the server. After calibration, the app will display a generic 3D model of the patient. If a band is not properly secured, the corresponding location will not activate in the app.
5) Manual Regulation Of Temperature:
Select Manual Mode in MySITIS App.
Select either HEAT or COOL.
Set the time interval.
6) Assisted Regulation Of Temperature:
Select Assist Mode in MySITIS App.

The system sets the HEAT and/or COOL time intervals. A suggested temperature value for each SITIS band appears.

The medical professional is able to receive trends and alerts through the MySITIS app, which detects when the patient is approaching hypothermic or hyperthermic levels based on the algorithm's analysis. These notifications are designed to appear as priority alerts. Users have the option to dismiss these warnings and extend the duration for which each region of the body is heated or cooled. However, the intensity of heating or cooling cannot be adjusted—only the time that each SITIS band remains active, intermittently active, or inactive. The system allows the selection of either Manual or Assist Mode. Each device is powered on by pressing its button, and additional SITIS bands can be added as necessary to accommodate the patient's needs.

Elements of the system include a deep learning framework, a HIPPA compliant API, flexible thermoelectric coolers, a first thermistor, a secondary thermistor, a humidity sensor, a heart rate sensor, and a respiration sensor. For software integration, it may be necessary to include a docker image containing the ensemble RNN, and secure cloud storage to store and further process anonymized healthcare data.

The device's alert system may be replaced with a physician's monitor or be displayed on a custom GUI on a laptop or PC. An app that is neither an Android or IOS mobile application may be implemented. The device may be coupled with heating films. The system may include infrared sensors to determine ambient temperature, and the ensemble RNN may use a convolutional neural network to visualize a sensor grid, allowing for spatial mapping and continuous monitoring of temperature across multiple points on the body to dynamically adjust thermoelectric cooling or heating modules for optimal thermoregulation.

In addition to thermoregulation in patients, the system can be adapted for tasks outside the medical field, including:
General Industry animal cooling and heating methods and tools.
Personalized cooling or heating garments methods and tools
General dog or cat or any household pet cooling and heating.
Heat stroke management methods and tools
Metal or plastic pipe heating or cooling tools.
General Furniture cooling or heating methods.
Perspiration control methods and tools.
Meditation methods and tools.
Stress management tools
Weight Loss Tools.

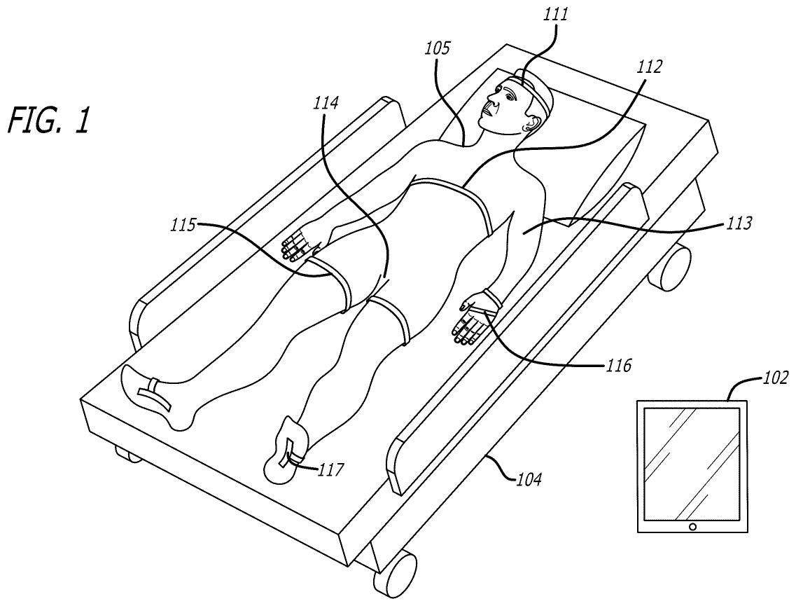

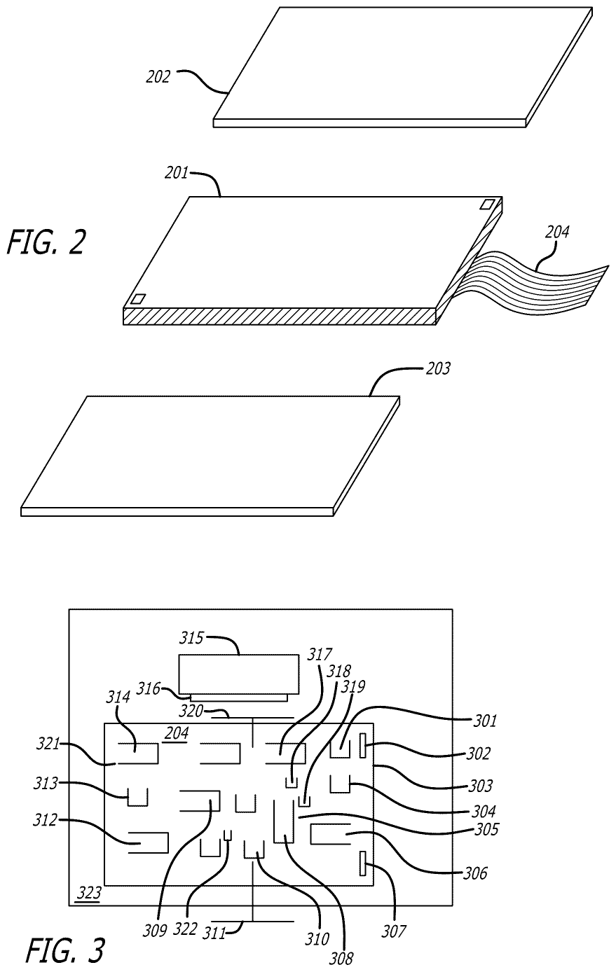

Turning to FIG. 1, a patient 105 is depicted in a patient bay customized for the thermoregulation system of the present invention, including the physical arrangement of additional monitoring devices, charging stations, and data synchronization equipment. The patient 105 rests on a hospital bed 104 and a tablet/display/computing device 102 is positioned proximate the bed 104. Seven locations are selected for temperature measurements, including the forehead 111, sternum 112, bicep 113, groin 114, inner thigh 115, palm 116, and sole 117. Temperature regulation is achieved in part using a flexible thermoelectric cooler 201 (FIG. 2) comprising a flexible PCM 202 and a secondary contact plate 203. Attached to the flexible cooler 201 and extending therefrom is a rigid-flex PCB 204. FIG. 3 is a schematic diagram of the PCB 204 of the flexible thermoelectric cooler 201. Dorsal to the flexible printed circuit board 204 is a rechargeable battery 315. Ventral to the flexible printed circuit board is a heart rate sensor 306, respiration sensor 303, thermistor 313, and an ambient temperature and humidity sensor 311.

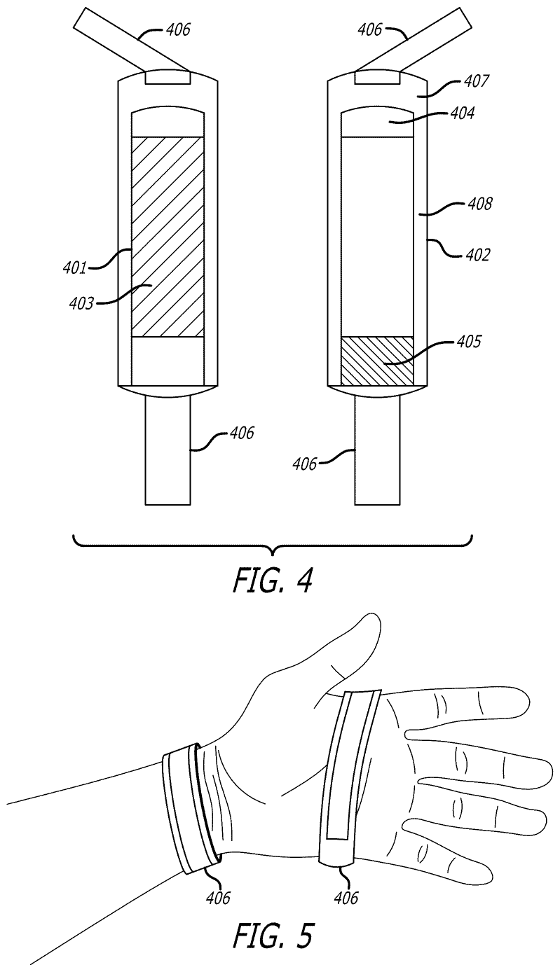

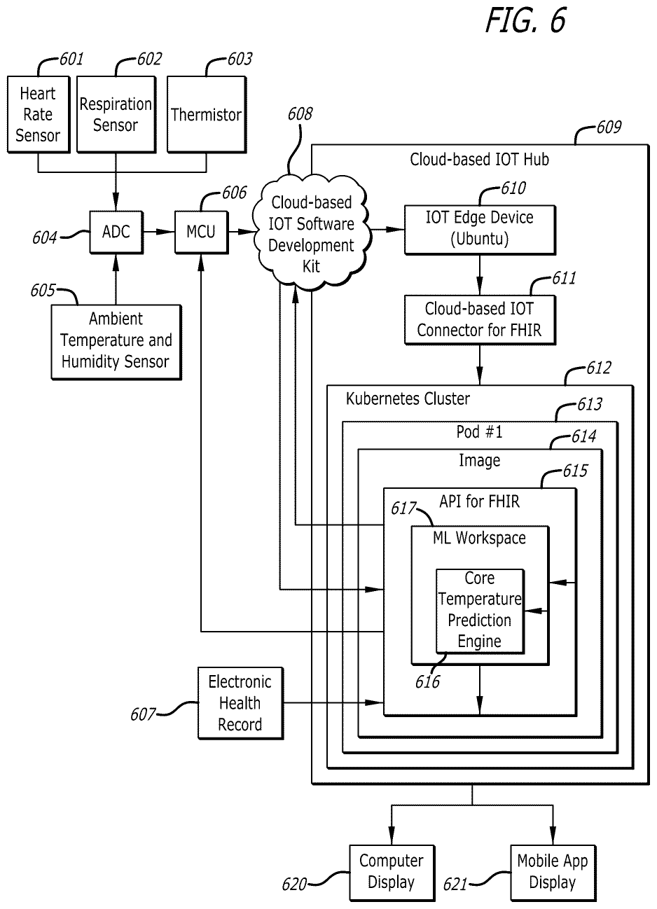

With reference to FIG. 4, the flexible thermoelectric cooler 201 is meticulously incorporated into the thermoelectric cooler section 403 and past a thermoelectric cooler plate section 401, to attain a seamless seal at the junction. Lateral to PCB slot 404 is a dome switch 408 and an RGB indicator light 407. At the opposite end from a PCB slot 404 is a battery slot 405. These hardware components are contained in the final outer casing 402 that has been cut for a thermoelectric cooler section 403 and a battery slot 405. Attached to the longitudinal extremities of the SITIS bands FIG. 4 are adjustable hook-and-loop straps 406, and best seen in FIG. 6.

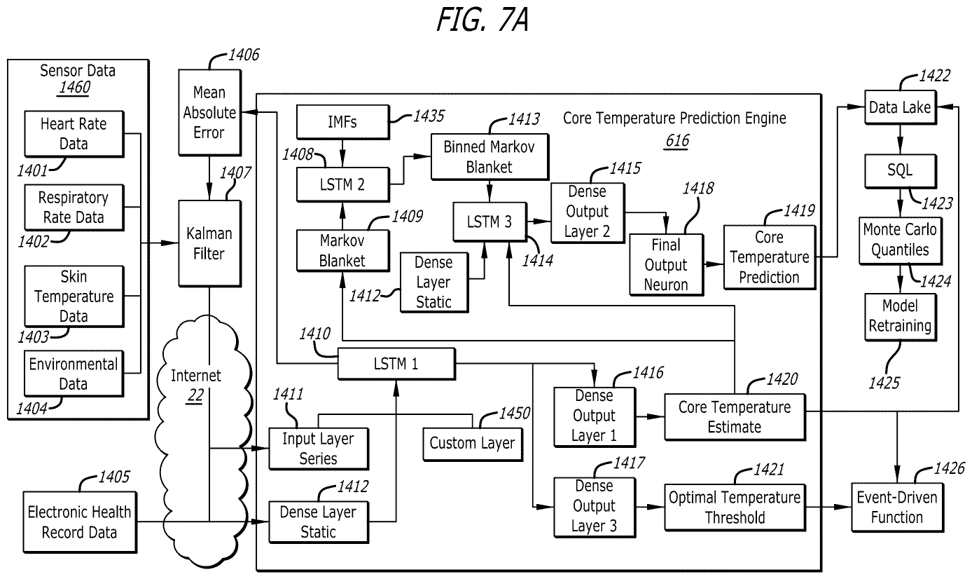

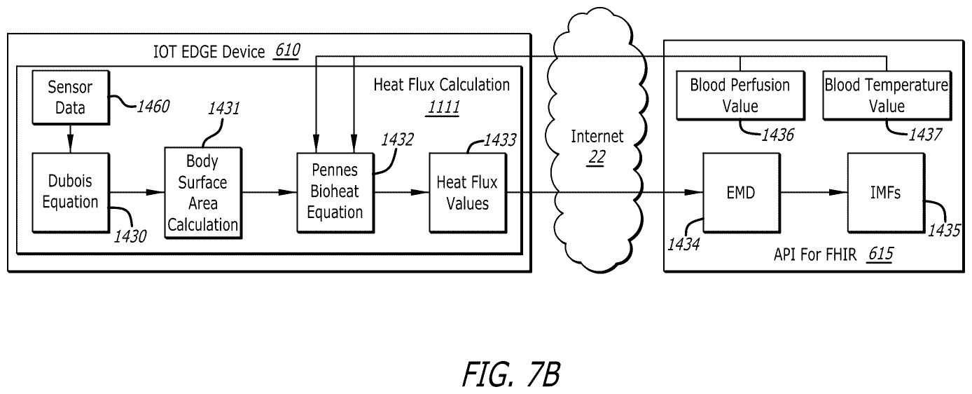

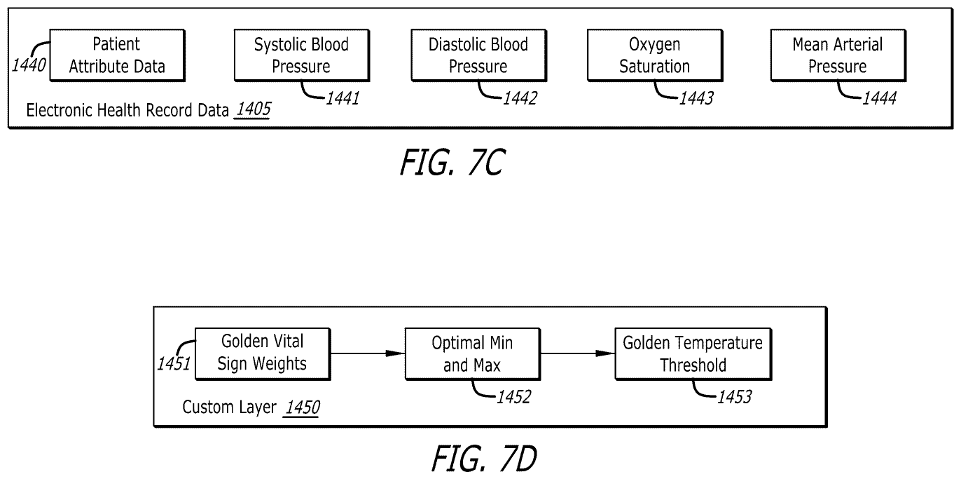

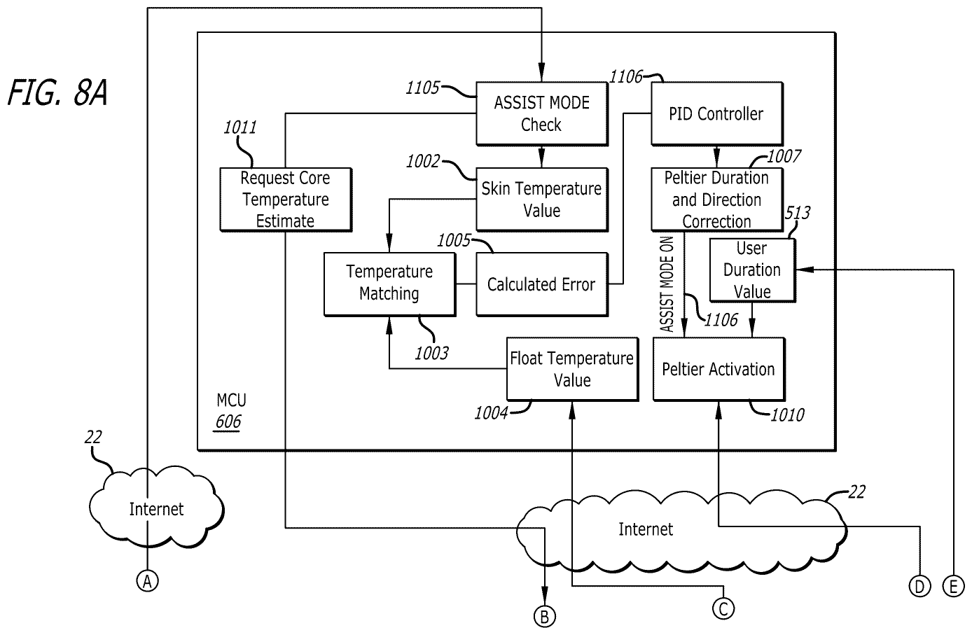

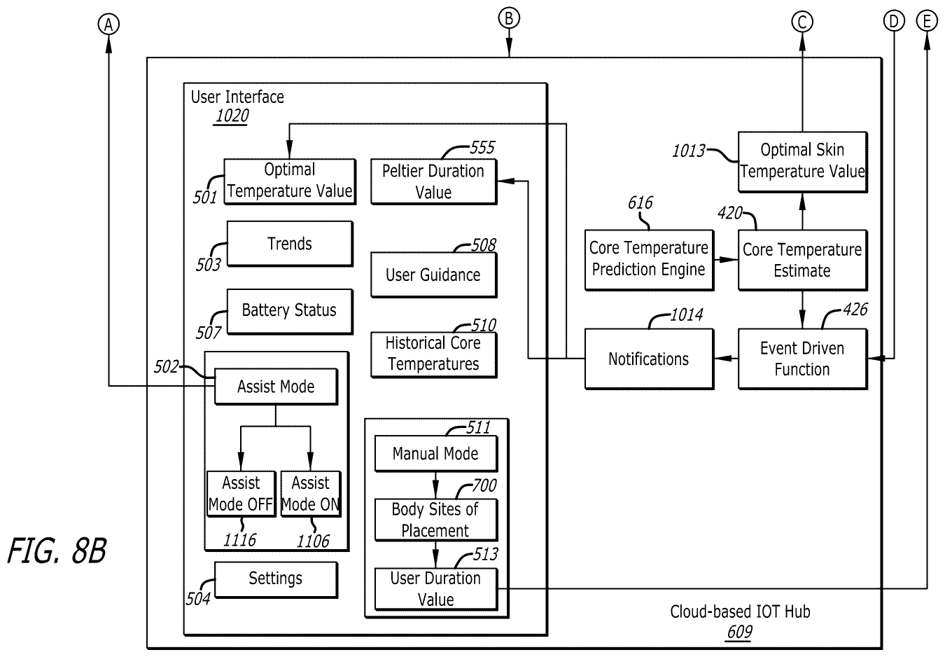

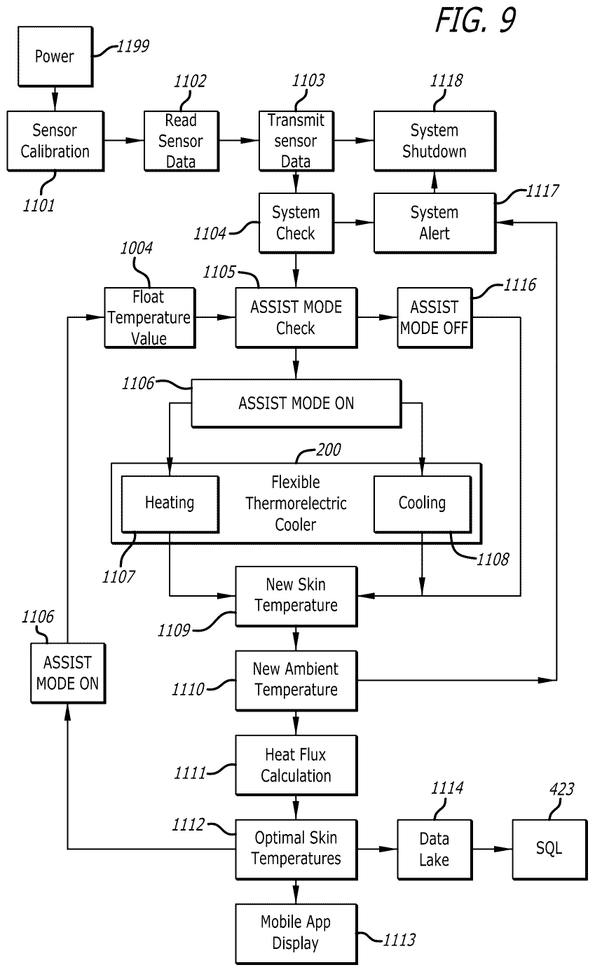

The software in SITIS100 initiates data collection by attaching SITIS bands via hook-and-loop straps 406 to a patient 105, illustrated by the placement of SITIS bands FIG. 7. With reference to FIG. 11, the dome switch 408 is pressed and held to power 1199 the SITIS bands FIG. 4. This begins a sensor calibration 1101 period to read sensor data 1102 and then begin to transmit sensor data 1103. A systems check 1004 is implemented to determine if sensor data is being transmitted. Data is gathered from a heart rate sensor 601 (FIG. 6), a respiration sensor 602, a thermistor 603, and an ambient temperature and humidity sensor 605 connected to an MCU 606 using an ADC 604, while an Electronic Health Record 607 is formatted to connect directly into an API for FHIR 615 via the internet 22. The MCU 606 uses a Kalman Filter 1407 to filter continuous heart rate data 1401, respiratory rate data 1402, and environmental data 1404 before offloading them to a cloud-based IoT edge device 610 via a cloud-based IoT software development kit 608. The cloud-based IoT edge device 610 uses environmental data 1404 and Dubois Equation 1430 for a body surface area calculation 1431 to use in Pennes Bioheat Equation 1432. The results are heat flux values 1433 in a heat flux calculation 1111 step from seven sites in the body sites of placement as illustrated in the placement of SITIS bands FIG. 7. The blood temperature value 1437 and the blood perfusion value 1436 are read from the Electronic Health Record 607 at a hospital institution using an API for FHIR 615. A mean absolute error 1406 step is implemented inside the IoT edge device 610 for time consistency. The seven newly calculated heat flux values 1433 are then sent to an API for FHIR 615 using a cloud-based IoT connector for FHIR 611. The API for FHIR 615 is created as an image 614 and is managed by any given pod #1 613 in Kubernetes cluster 612 to anonymize data for a human core temperature prediction engine 616.

The core temperature prediction engine 616 is registered to a ML workspace 617 and is deployed on a Kubernetes cluster 612. It uses 3 individual LSTM models to extract features from time series Electronic Health Record Data 405. The inputs of LSTM 1 1410 are systolic blood pressure 1441, diastolic blood pressure 1442, oxygen saturation 1443, mean arterial pressure 1444, heart rate data 1401, respiratory rate data 1402 and the output is a core temperature estimate 1420 and an optimal temperature threshold 11421. The inputs of a Markov Blanke 1409 is a core temperature estimate 1420. The inputs of LSTM 2 1408 are two averaged IMFs 1435 derived from an EMD 1434 process, and the output from a Markov Blanket 1409. The outputs of the LSTM 2 408 are used as inputs for a binned Markov Blanket 1413. The outputs of the binned Markov Blanket 1413 are used as inputs for LSTM 3 1414. A dense layer static 1412 is used to extract features from the nontime-series patient attribute data 1440 of height, weight, age, sex, race, and time of day in Electronic Health Record Data 1405. The output from all three models are concatenated and used in the dense output layer 2 1415. The final output neuron 1418 performs the final regression task and returns the core temperature prediction 1419. The core temperature prediction 1419 is continuously stored in a data lake 1422 and extracted into an SQL 1423 to undergo Monte Carlo quantiles 1424 in a ML workspace 617 for model retraining 1425.

The core temperature estimate 1420 and the optimal temperature threshold 1421 pertaining to each unique patient is found by a single model, LSTM 1 1410, consisting of three LSTM layers, an input layer series 1411, a dense layer static 1412, a custom layer 1450. The input layer series 1411 is composed of time series data from systolic blood pressure 1441, diastolic blood pressure 1442, oxygen saturation 1443, mean arterial pressure 1444, heart rate data 1401 and respiratory rate data 1402 to target a core temperature feature. The dense layer static 1412 is used to process non-time series patient attribute data 1436 of height, weight, age, sex, race, and time of day in Electronic Health Record data 1405. A custom layer 1450 containing golden vital sign weights 1451 are followed by learnable optimal min and max 1452 values to penalize inferences made outside of an initial golden temperature threshold 1453. A dense output layer 1 1416 returns the results containing the core temperature estimate 1420 and a second dense output layer 3 1417 returns the results containing the optimal temperature threshold 1421. Only the core temperature estimate 1420 is reshaped and used as an input in the core temperature prediction engine 616.

In assist mode 502, the MCU 606 receives instructions from the user interface 1020 via the internet 22 based on the assist mode on 1106 or assist mode off 1116 position, as performed by an assist mode check 1005 loop. A cloud-based software development kit 608 in the MCU 606 using an IoT hub connection string is used for direct communication to an IoT Hub 609 and initiates a request core temperature estimate 1011 step. This prompts the API for FHIR 615 in a Kubernetes cluster 612 to request an inference from the core temperature prediction engine 616. The core temperature prediction engine 616 responds with 3 values. The first value is the output from the LSTM 2 1408. An API for FHIR 615 processes the values from the LSTM 2 1408 and converts them into an optimal skin temperature value 1013, removing any patient identifiers. The MCU 606 expects to receive this value as a float temperature value 1004. The incoming values enter a temperature matching 1003 step, where the incoming temperatures are aligned to the placement of SITIS bands with the first place being the forehead 111 and the seventh place being the soles of the feet 117.

After matching the vector, a calculated error 1005 is performed in the MCU 606 for the difference between a current skin temperature value 1002 and the float temperature value 1004. The PID controller 1006 receives each calculated error 1005 sequentially and outputs a matrix of values corresponding to a new peltier duration and direction correction value 1007 for each of the 7. The peltier duration and direction correction 1007 value from the PID controller 1006 are used and peltier activation 1010 is initialized. This triggers the MCU 606 to supply or negate voltage from a battery 315 to a flexible thermoelectric cooler 201 and controlled by a driver for thermoelectric cooler 317. This begins a non-intermittent heating 1107 and an intermittent cooling 1108 process via flexible thermoelectric coolers 201 to regulate the user's skin temperature value 1002 in a nested loop. The loop continues to activate the flexible thermoelectric coolers 201 as long as assist mode on 1106 persists in the frontend. New skin temperature 1109 and new ambient temperature 1110 values are monitored and displayed every 10 minutes in the display 737. While in assist mode off 1116, the system checks for a user duration value 513 from the user interface 1020 in the frontend, and proceeds to use it for peltier activation 1010. This is configured in a manual mode 511 in the user interface 1020.

A system alert 1117 is implemented using a buzzer 319 and is triggered if the MCU 606 determines if new skin temperature 1109 reaches 10% of a predefined skin temperature value 1002. Upon reaching 5%, the MCU 606 triggers a system shutdown 1118 for the thermoelectric cooler 2000. A backup relay 321 is implemented to detect high mechanical heat changes in the skin and to disconnect power to the flexible thermoelectric cooler 201 without input from the MCU 606. New skin temperature 1109 and new ambient temperature are continuously monitored. Regardless of assist mode, the core temperature estimates are sent to a data lake 1422 parallel to an event-driven function 1426 for event-driven actions. These actions send the estimates to the backend for graph creation, while also updating the user interface 1020, which includes a computer display 620, a mobile app display 621 in a tablet 102, as well as the IoT hub 609. The computer display 620 and mobile app display 621 show real-time data, and server-side Notifications 1014 are pushed to users through a mobile application FIG. 5 to alert if core temperature estimates 420 fall outside the optimal temperature threshold 421. The mobile app display 621 includes modules for optimal temperature value 501, assist mode 502, user duration value 513, trends 503, settings 504, battery status 507, user guidance 508 and historical core temperatures 510.

The foregoing descriptions and depictions represent the inventor's best mode of carrying out the invention. While certain embodiments have been described for illustrative purposes, the invention is not limited to those specific components, values, etc. A person of ordinary skill in the art will readily recognize modifications, additions, and variations that are properly deemed to fall within the scope of the invention. Thus, absent expressed limitation, the invention is properly deemed to include all such variations consistent with the ordinary meaning of the terms of the claims appended hereto.

I claim:

1. An method for controlling a core body temperature (CBT) of a user, comprising:
applying a wearable apparatus to the user, the wearable apparatus including a sensor;
collecting biometric data from the user, including at least one of heart rate, breathing rate, and skin temperature, via the wearable apparatus;
collecting environmental data proximal said user, said environmental data including at least one of ambient temperature and humidity;
recalling Electronic Health Record data from the user, including at least one of heart rate, systolic blood pressure, diastolic blood pressure, respiratory rate, oxygen saturation, body temperature, mean arterial pressure, and lactate level;
processing the biometric data, environmental data, and Electronic Health Record data through a predictive algorithm, wherein the predictive algorithm analyzes multi-factor inputs to calculate a predicted core body temperature for the user; and
determining a prediction of a future core body temperature based on the processed data.

2. The method of claim 1, further comprising outputting a recommendation to the user when the predicted future core body temperature exceeds a predefined safe threshold, the recommendation including at least one inbuilt cooling strategy via a wearable device equipped with a flexible thermoelectric cooler.

3. The method of claim 1, further comprising outputting a recommendation to the user when the predicted future core body temperature falls below a predefined safe threshold, the recommendation including at least one inbuilt warming strategy via a wearable device equipped with a flexible thermoelectric warmer.

4. The method of claim 1, wherein the predictive algorithm further incorporates personalized user data, including at least one of height, weight, medication, age, race, sex, actual time, and historical temperature data of the user.

5. The method of claim 1, wherein the predictive algorithm employs an ensemble of three long short-term memory models executed by a processor, the long short-term memory models configured to refine and improve the prediction accuracy of the predicted core body temperature over time by processing ongoing biometric, environmental data and Electronic Health Record data.

6. The method of claim 5, wherein the ensemble of three long short-term memory models are selected from a group consisting of supervised learning, unsupervised learning, and reinforcement learning, and wherein the models are trained based on user-specific temperature patterns to optimize prediction accuracy.

7. The method of claim 2, wherein the outputting step includes providing a real-time alert to the user if the predicted core body temperature exceeds a threshold indicative of hyperthermia, and wherein the recommendation includes an inbuilt cooling strategy based on the predicted core body temperature and a human input.

8. The method of claim 2, wherein the outputting step includes providing a real-time alert to the user if the predicted core body temperature exceeds a threshold indicative of hypothermia, and wherein the recommendation includes an inbuilt warming strategy based on the predicted core body temperature and a human input.

9. The method of claim 1, wherein the wearable device is selected from a group consisting of a flexible thermoelectric cooler, a medical-grade heart rate sensor, a medical-grade respiratory sensory, a medical-grade thermistor or other biometric sensing device capable of continuously monitoring the user's vital signs and movement to assess core body temperature.

10. The method of claim 1, further comprising the storage of the collected biometric and environmental data, along with the predicted core body temperature, in a cloud-based data repository, wherein the stored data is accessible by authorized third-party entities through secure interfaces, enabling the remote retrieval, analysis, monitoring, or research of user-specific thermoregulatory patterns and system performance.
